(19) United States
(12) Patent Application Publication
(10) Pub. No.: US 2009/0299412 A1
(43) Pub. Date: Dec. 3, 2009

(54) PERCUTANEOUS FACET JOINT FUSION SYSTEM AND METHOD

(75) Inventor: James F. Marino, La Jolla, CA (US)

Correspondence Address: MINTZ, LEVIN, COHN, FERRIS, GLOVSKY AND POPEO, P.C, ONE FINANCIAL CENTER, BOSTON, MA, 02111, US

(73) Assignee: TRINITY ORTHOPEDICS, San Diego, CA (US)

(21) Appl. No.: 12/278,127

(22) PCT Filed: Feb. 2, 2007
(86) PCT No.: PCT/US07/02847

§ 371 Date: Jul. 21, 2009

Related U.S. Application Data
(60) Provisional application No. 60/764,935, filed on Feb. 2, 2006.

(51) Int. Cl. A61B 17/70; A61B 17/88; A61B 17/00

(52) U.S. Cl. 606/246; 606/279; 606/80

(57) ABSTRACT

Apparatus and methods for percutaneously fusing or stabilizing a bony surface.

### CROSS REFERENCE TO RELATED APPLICATION

This invention is related to Provisional Patent Application No. 60/764,935, filed Feb. 2, 2006, Attorney Docket Number TO008US, and entitled “Percutaneous Facet Joint Fusion Methodology” which is hereby incorporated by reference for its teachings.

### BACKGROUND

1. Field of the Invention

The invention relates generally to orthopedic boney fusion and stabilization systems and methods, and more particularly, to percutaneous fusion and stabilization systems and methods.

2. Description of Related Art

It is desirable to provide a percutaneous fusion and stabilization system and method that limits or prevent the risks of nerve injury or epineural fibrosis. The present invention provides such a system and method.

### BRIEF DESCRIPTION OF THE DRAWINGS

The features, objects, and advantages of the present invention will become more apparent from the detailed description set forth below when taken in conjunction with the drawings in which like reference characters identify correspondingly throughout and wherein:

FIG. 1A is a simplified sagittal view of a vertebrae pair;

FIG. 1B is a simplified, sectional coronal view a vertebrae;

FIG. 2A is a simplified coronal view of the vertebrae pair including a guide pin and a support sleeve, the guide pin being inserted into a facet joint between the vertebra pair in accordance with an embodiment of the present invention;

FIG. 2B is a simplified sagittal view of the vertebrae pair including the guide pin and the support sleeve, the guide pin being inserted into the facet joint between the vertebra pair as shown in FIG. 2A;

FIG. 2C is a simplified posterior view of the vertebrae pair including the guide pin and the support sleeve, the guide pin being inserted into the facet joint between the vertebra pair as shown in FIG. 2A;

FIG. 2D is a simplified isometric view of the vertebrae pair including the guide pin and the support sleeve, the guide pin being inserted into the facet joint between the vertebra pair as shown in FIG. 2A;

FIG. 3A is a simplified isometric view of the vertebrae pair shown in FIG. 2D further including an obturator and a cannula inserted over the guide pin and the support sleeve, the obturator being advanced toward the facet joint between the vertebra pair to create a pathway to the facet joint in accordance with an embodiment of the present invention;

FIG. 3B is a simplified isometric view of the vertebrae pair where the obturator and guide sleeve have been removed leaving the guide pin inserted into the facet joint with the cannula over the guide pin in accordance with an embodiment of the present invention;

FIG. 3C is a simplified isometric view of the vertebrae pair shown in FIG. 3B further including a cannulated reamer inserted over the guide pin and within the cannula, the reamer being operatively advanced into the facet joint to form a bore in the facet joint in accordance with an embodiment of the present invention;

FIG. 4A is a simplified isometric view of the vertebrae pair where the cannulated reamer has been removed leaving the guide pin inserted in the bored facet joint and the cannula over the guide pin in accordance with an embodiment of the present invention;

FIG. 4B is a simplified isometric view of the vertebrae pair shown in FIG. 4A where a fusion construct has been inserted into the facet joint bore in accordance with an embodiment of the present invention;

FIG. 5A is a simplified isometric view of the vertebrae pair shown in FIG. 4B where a fusion construct has been inserted into the left and right facet joints and a pedicle based fixation construct has been placed on the left and right side of the vertebrae pair in accordance with an embodiment of the present invention;

FIG. 5B is a simplified posterior view of the vertebrae pair shown in FIG. 5A where a fusion construct has been inserted into the left and right facet joints and a pedicle based fixation construct has been placed on the left and right side of the vertebrae pair in accordance with an embodiment of the present invention;

FIG. 5C is a simplified coronal view of the vertebrae pair shown in FIG. 5A where a fusion construct has been inserted into the left and right facet joints and a pedicle based fixation construct has been placed on the left and right side of the vertebrae pair in accordance with an embodiment of the present invention; and

FIG. 5D is a simplified sagittal view of the vertebrae pair shown in FIG. 5A where a fusion construct has been inserted into the left and right facet joints and a pedicle based fixation construct has been placed on the left and right side of the vertebrae pair in accordance with an embodiment of the present invention.

### DETAILED DESCRIPTION

Throughout this description, embodiments and variations are described for the purpose of illustrating uses and implementations of the invention. The illustrative description should be understood as presenting examples of the invention, rather than as limiting the scope of the invention.

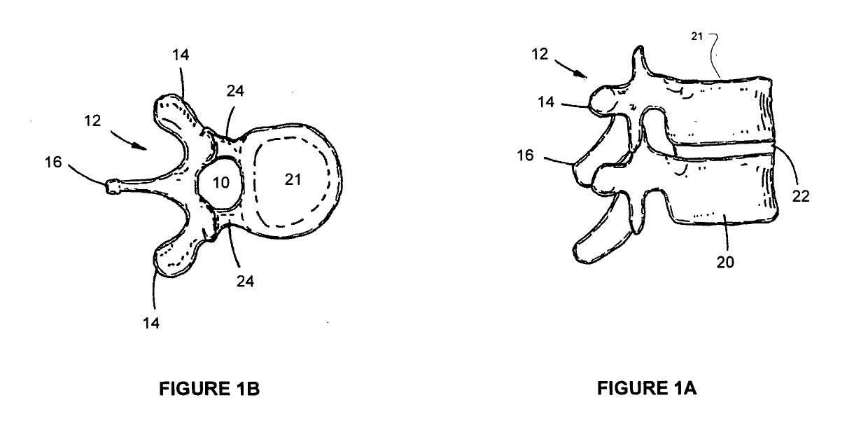

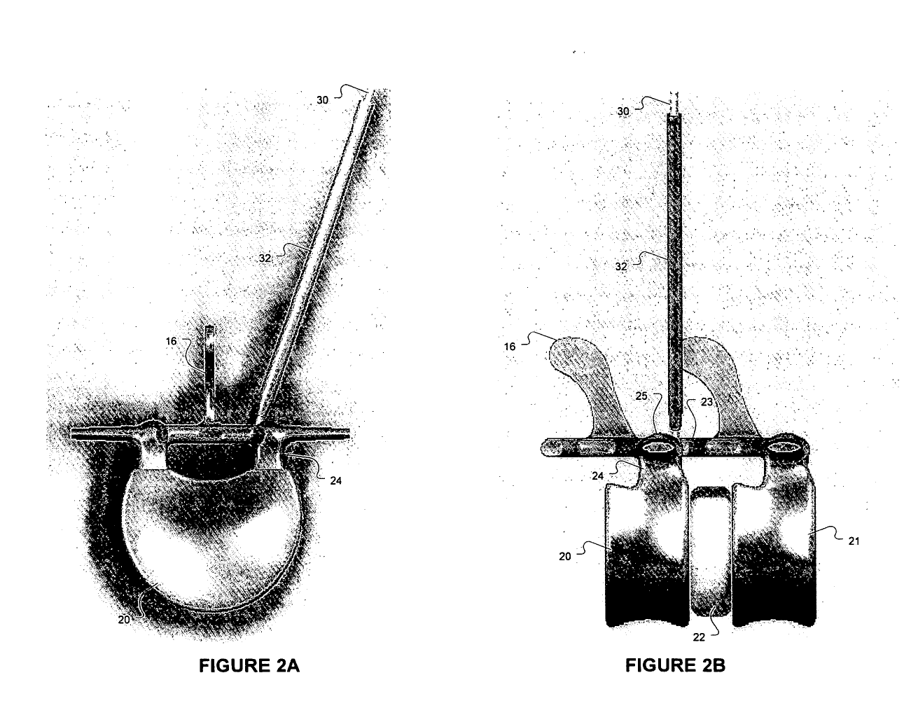

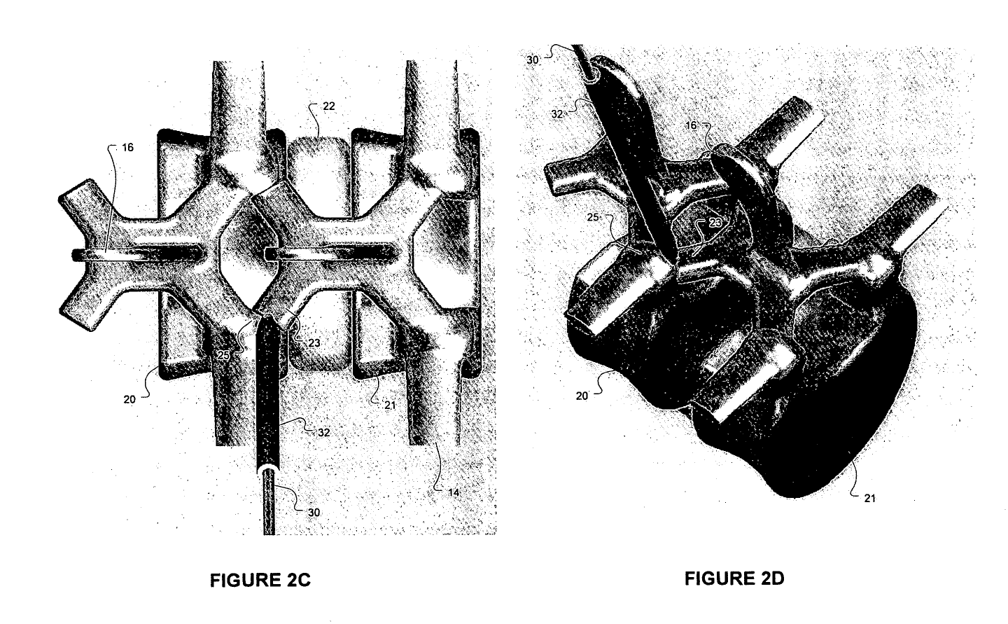

FIG. 1A is a simplified sagittal view of a vertebrae pair 20, 21. FIG. 1B is a simplified, sectional coronal view of the vertebrae 21 of the vertebrae pair shown in FIG. 1A. Each vertebra 20, 21 includes lamina 12, transverse processes 14, a spinous process 16, central canal 10, and pedicles 24. A disc 22 comprised of an annulus and disc nucleus (not shown) is located between the vertebrae pair 20, 21 where the vertebrae pair 20, 21 and disc 22 form a coupled articulated jointed bony interface. Due to disc degeneration, expulsion, annulus tears, or other conditions, the spinal cord that passes through the central canal 10 may become compressed causing patient discomfort. It may be desirable to modify or fix the spatial relationship between the vertebrae pair 20, 21. FIGS. 2A to 5D present various apparatus and methods for fusing the vertebrae pair 20, 21 via the facet joints between the vertebrae pair 20, 21 where the facet joints between a vertebrae pair 20, 21 form an uncoupled articulated jointed bony interface.

FIG. 2A is a simplified coronal view, FIG. 2B is a simplified sagittal view, FIG. 2C is a simplified posterior view, and FIG. 2D is an isometric view of the vertebrae pair 20, 21 including a guide pin or wire 30 and a support sleeve 32 in accordance with an embodiment of the present invention. In this embodiment, the guide pin 30 is inserted at a posterior, lateral angle from the coronal view and normal to the vertebrae 20 from the sagittal view. The guide pin extends into the vertebrae 20, vertebrae 21 facet joint. The facet joint is formed by vertebrae 20 superior process 25 and vertebrae 21 inferior process 23 (as shown in FIG. 2C). In addition in an embodiment a support sleeve 32 may be inserted over the guide pin 30. The support sleeve 32 may be a thin walled cannula in an embodiment of the present invention.

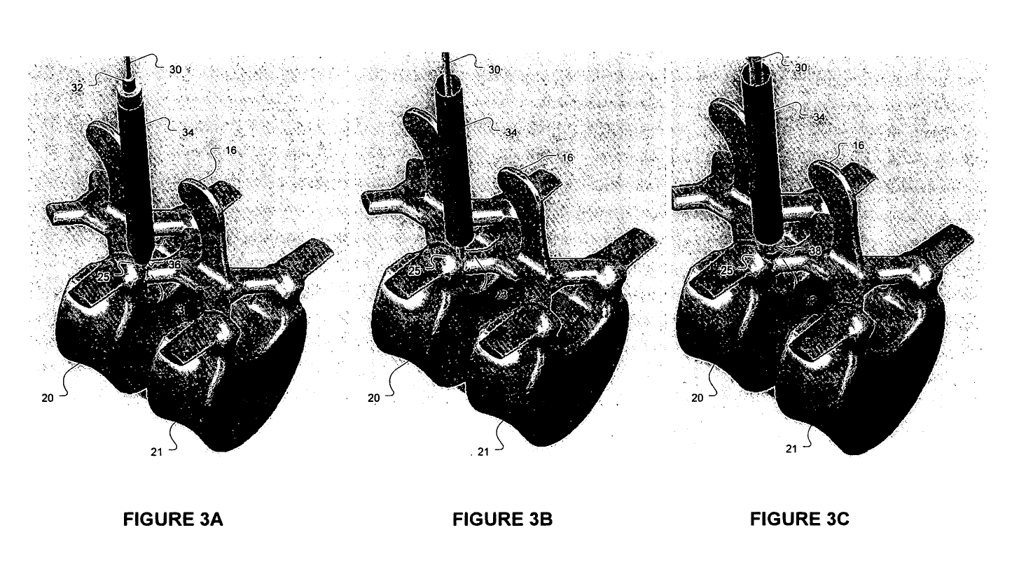

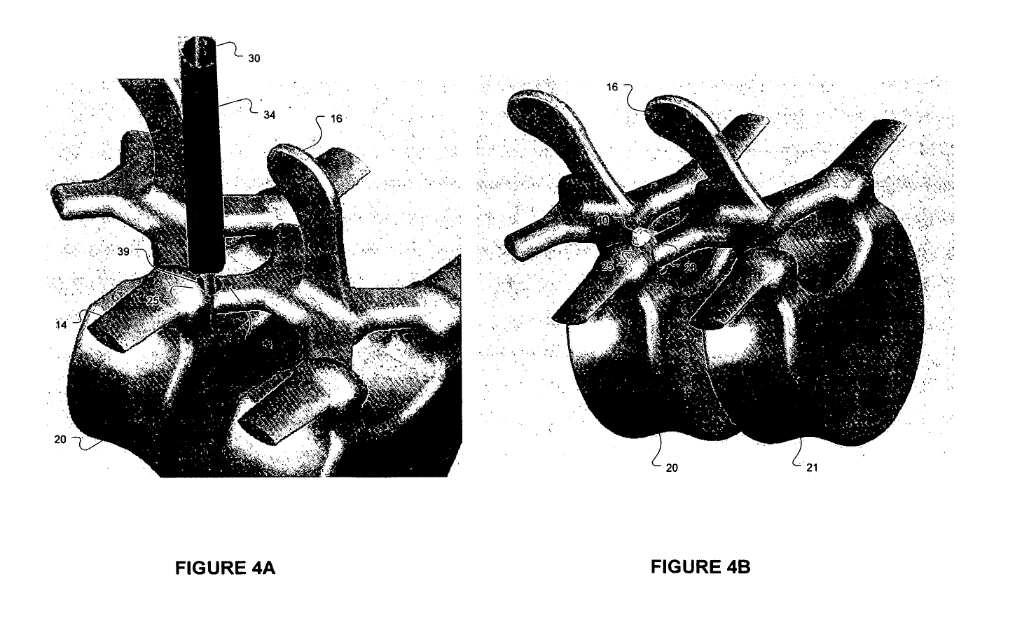

FIG. 3A is a simplified isometric view of the vertebrae pair 20, 21 shown in FIG. 2D further including an obturator 36 and cannula 34 inserted over the guide pin 30 and support sleeve 32. In an embodiment the obturator 36 may be advanced toward to facet joint 23, 25 to create a tissue pathway to the facet joint. FIG. 3B is a simplified isometric view of the vertebrae pair 20, 21 where the obturator 36 and guide sleeve 32 have been removed leaving the guide pin 30 inserted into the facet joint with the cannula 34 over the guide pin 30. FIG. 3C is a simplified isometric view of the vertebrae pair 20, 21 shown in FIG. 3B further including a cannulated reamer 38 inserted over the guide pin 30 and within the cannula 34. In an embodiment, the reamer 38 may be operatively advanced into the facet joint to form a bore in the facet joint 23, 25. In an embodiment the reamer 38 may have about a 5 mm diameter and about an 8 mm depth stop. In this embodiment, the reamer 38 may be used to form an approximately 10 mm deep, 5 mm in diameter bore (39 shown in FIG. 4A) in the facet joint 23, 25, the bore 39 axis being approximately normal to the coronal plane of vertebrae 20. In this embodiment the cannula 34 may have a diameter of about 8.5 mm.

FIG. 4A is a simplified isometric view of the vertebrae pair 20, 21 where the cannulated reamer 38 has been removed leaving the guide pin 30 with cannula 34 inserted in the bored facet joint in accordance with an embodiment of the present invention. FIG. 4B is a simplified isometric view of the vertebrae pair 20, 21 shown in FIG. 4A where a fusion construct 40 has been inserted into the facet joint. In an embodiment the fusion construct may be cannulated so it may be inserted over the guide pin 30 and through the cannula 34 into the facet joint bore. The fusion construct 40 may have a diameter greater than the bore diameter. In another embodiment the fusion construct is not cannulated. In this embodiment the guide pin 30 may be removed prior to the fusion construct 40 insertion. The fusion construct may be advanced into the facet joint bore via the cannula 34. In an embodiment the fusion construct 40 may include bone.

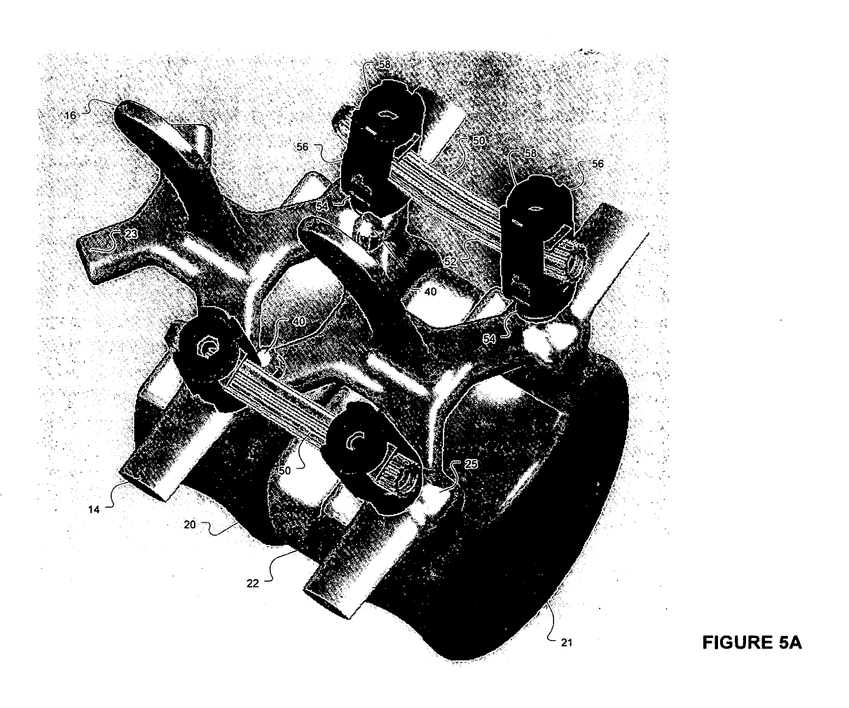

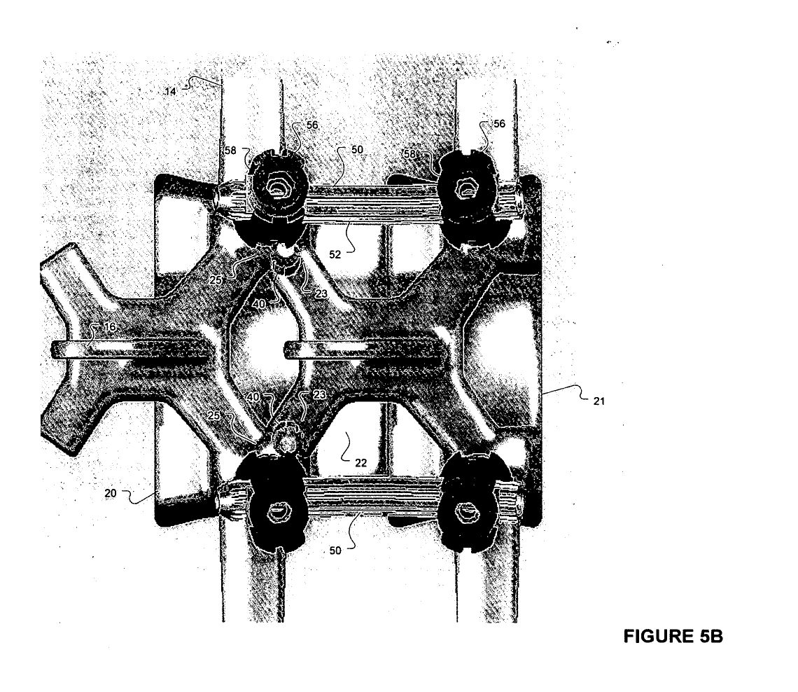

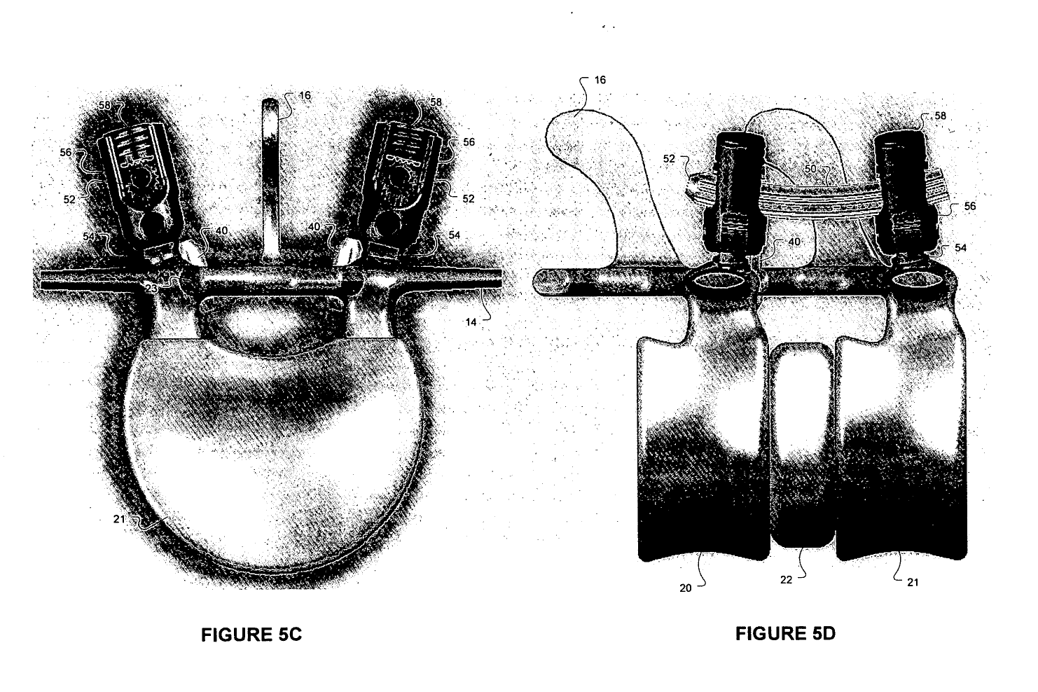

In an embodiment additional fixation constructs may be employed to aid facet joint fusion with the fusion construct 40. FIG. 5A is a simplified isometric view, FIG. 5B is a simplified posterior view, FIG. 5C is a simplified coronal view, and FIG. 5D is a simplified sagittal view of the vertebrae pair shown in FIG. 4B where a fusion construct 40 has been inserted into the left and right facet joints and a pedicle based fixation construct 50 has been placed on the left and right side of the vertebrae pair 20, 21 in accordance with an embodiment of the present invention.

In an embodiment the fixation construct 50 includes two pedicles screws 54, receiving members 56, two rod locking caps 58, and a serrated rod 52. In an embodiment each pedicle screw 54 is inserted into a pedicle 24 of the vertebrae 20, 21 along the same side of the pair 20, 21. In an embodiment each pedicle screw 54 is uni-axially coupled to a rod receiving member 56. After each pedicle screw, receiving member 56 combination is inserted into a pedicle, a rod may be placed into the receiving members 56. After the desired disc 22 distraction or compression is achieved locking caps 58 may secured against the rod 52 ends to fixate a side of the vertebrae 20, 21 pair. As noted such fixation may aid facet joint fusion in an embodiment. In an embodiment a fusion construct 40 is placed in the left and right facet joints and a fixation construct 50 is also placed on the left and right side of the vertebrae pair 20, 21.

While this invention has been described in terms of a best mode for achieving the objectives of the invention, it will be appreciated by those skilled in the art that variations may be accomplished in view of these teachings without deviating from the spirit or scope of the present invention. For example, the inferior vertebrae 20 may be the sacrum and the superior vertebrae 21 the adjacent vertebrae, L5 in humans. The present invention may also be employed in any bony interface to promote fusion at the bony interface.

1.-20. (canceled)

21. A percutaneous spinal stabilization system, comprising:
a percutaneous delivery system comprising:
a guide wire; and
a cannulated obturator,
wherein the guide wire inserts into a first facet joint between an inferior vertebra and a superior vertebra and wherein the cannulated obturator passes over the guide wire and creates a tissue pathway to the first facet joint;
a fusion construct, wherein the fusion construct passes over the guide wire through the tissue pathway into the first facet joint; and
a fixation construct comprising:
a first and second fixation element,
a first and second receiving member, and
a rod spanning between the first and second receiving members,
wherein the first fixation element inserts into a first pedicle of the superior vertebra and the second fixation element inserts into a first pedicle of the inferior vertebra.

22. The system of claim 21, wherein the percutaneous delivery system further comprises a cannulated reamer, wherein the reamer passes over the guide wire and creates a bore in the first facet joint.

23. The system of claim 22, wherein the fusion construct inserts within the bore in the first facet joint.

24. The system of claim 23, wherein the fusion construct has a diameter that is larger than the bore diameter.

25. The system of claim 21, wherein the fusion construct comprises bone.

26. The system of claim 21, wherein the fusion construct is cannulated.

27. The system of claim 21, further comprising a second fusion construct, wherein the second fusion construct passes over a second guide wire inserted into a second bore formed in a second facet joint between the inferior and superior vertebrae.

28. The system of claim 27, wherein the second fusion construct comprises bone.

29. The system of claim 27, wherein the second fusion construct is cannulated.

30. The system of claim 27, further comprising a second fixation construct comprising:
a third and fourth fixation element,
a third and fourth receiving member, and
a second rod spanning between the third and fourth receiving members,
wherein the third fixation element inserts into a second pedicle of the superior vertebra and the fourth fixation element inserts into a second pedicle of the inferior vertebra.

31. A method for percutaneously stabilizing the spine comprising:
creating a percutaneous pathway to a first facet joint between an inferior vertebra and a superior vertebra;
inserting a fusion construct within the first facet joint through the percutaneous pathway;
inserting a fixation construct, the fixation construct comprising:
a first and second fixation element,
a first and second receiving member,
a first and second set screw and a rod spanning between the first and second receiving members,
wherein the first fixation element inserts into a first pedicle of the superior vertebra and the second fixation element inserts into a first pedicle of the inferior vertebra.

32. The method of claim 31, further comprising inserting a guide wire into the first facet joint.

33. The method of claim 32, further comprising inserting a cannulated obturator over the guide wire and creating a tissue pathway to the first facet joint.

34. The method of claim 33, further comprising inserting a cannulated reamer over the guide wire and toward the first facet joint creating a bore in the first facet joint.

35. The method of claim 34, wherein inserting the fusion construct comprises inserting the fusion construct within the bore in the first facet joint.

36. The method of claim 31, wherein the fusion construct comprises bone.

37. The method of claim 31, wherein the fusion construct is cannulated.

38. The method of claim 31, further comprising:
creating a second percutaneous pathway to a second facet joint between the inferior vertebra and the superior vertebra; and
inserting a second fusion construct into a second bore formed in the second facet joint through the second percutaneous pathway.

39. The method of claim 38, wherein the second fusion construct comprises bone.

40. The method of claim 38, wherein the second fusion construct is cannulated

41. The method of claim 38, further comprising inserting a second fixation construct comprising:
a third and fourth fixation element,
a third and fourth receiving member, and
a second rod spanning between the third and fourth receiving members,
wherein the third fixation element inserts into a second pedicle of the superior vertebra and the fourth fixation element inserts into a second pedicle of the inferior vertebra.

42. A method for percutaneously stabilizing the spine comprising:
inserting a guide wire into a first uncoupled articulated jointed bony interface;
advancing a cannulated obturator over the guide wire and toward the first uncoupled articulated jointed bony interface;
creating a tissue pathway in bony elements of the articulated jointed bony interface using a cannulated reamer;
inserting a fusion construct within the first uncoupled articulated jointed bony interface through the tissue pathway; and
inserting a fixation construct, the fixation construct comprising:
a first and second fixation element,
a first and second receiving member,
a first and second set screw and a rod spanning between the first and second receiving members.
